(19) United States
(12) Patent Application Publication
(10) Pub. No.: US 2025/0264463 A1
(43) Pub. Date: Aug. 21, 2025

(54) LATERAL FLOW ASSAY TEST STRIPS AND SYSTEMS, AND METHODS OF USE THEREOF

(71) Applicant: CARDIAI TECHNOLOGIES LTD., Calgary, Alberta (CA)

(72) Inventors: Anmol KAPOOR, Calgary, Alberta (CA); Vivek Krishna PULAKAZHI VENU, Calgary, Alberta (CA); Shabnam VALADAN, Calgary, Alberta (CA); Sidharth Singh BHINDER, Calgary, Alberta (CA); Raja Balraj SINGH, Calgary, Alberta (CA)

(21) Appl. No.: 18/857,783

(22) PCT Filed: Apr. 18, 2023
(86) PCT No.: PCT/CA2023/050522

§ 371 Date: Oct. 17, 2024

Related U.S. Application Data
(60) Provisional application No. 63/332,522, filed on Apr. 19, 2022.

(51) Int. Cl. G01N 33/543; B82Y 15/00; G01N 33/68

(52) U.S. Cl. CPC G01N 33/54388; G01N 33/54346; G01N 33/6887; B82Y 15/00; G01N 2333/58

(57) ABSTRACT

A lateral flow assay test strip, device, methods, system and kit for detecting a target analyte in a test sample. The test strip has a sample receiving zone, a reaction zone comprising a conjugatively-labeled detector reagent that specifically binds to the target analyte and a labeled control reagent having a control-specific label, a detection zone comprising a test line region with an immobilized detector capture reagent that specifically binds to the target analyte and a control line region having an immobilized control capture reagent that specifically binds to the labeled control reagent, and an absorbent zone. The conjugatively-labeled detector reagent has an analyte-specific label covalently-conjugated to an analyte binding reagent and such analyte-specific label is of a larger size or a greater signal sensitivity than the control-specific label, and the analyte-specific label has a different detectable characteristic feature than the control-specific label.

### CROSS-REFERENCE TO RELATED APPLICATION

This application claims priority to and benefit from U.S. Patent Application Ser. No. 63/332,522 filed on Apr. 19, 2022, which is hereby incorporated by reference in its entirety.

### TECHNICAL FIELD

The present disclosure relates generally to lateral flow assay test strips, devices, systems, kits, and methods for detecting one or more target analytes in a test sample, and in particular, to a lateral flow assay test strips employing labels with different sizes and detectable characteristic features for a test line region and a control line region.

### BACKGROUND

Lateral flow assay strips are widely used in many different areas of analytical chemistry and medicine and are available for testing for a wide variety of analytes or compounds of interest in a given test sample, such as an antigen, a toxin, a hormone, or a specific biomarker in blood or plasma.

However, currently available lateral flow assay strips are not ideal for detecting the presence of certain analytes, for example due to their low concentrations, such as many cardiac markers. Devices and methods having improved sensitivity and other characteristics are thus needed.

Furthermore, currently available test strips use labels having the same detectable characteristic features (e.g. color) for both their test lines and control lines. Although visual inspection of such lateral flow assay strips may provide qualitative results and may be able to provide quantitative measurement to a certain degree based on visual determination of the intensity of signals, such visual inspection does not provide accurate measurements and is also prone to interpretation errors.

It is therefore an object to provide improved lateral flow assay strips, devices, and systems.

### SUMMARY

The present disclosure relates to lateral flow assay test strips, devices, systems, kits, and methods for detecting one or more target analytes in a test sample.

According to one aspect of this disclosure, there is provided a lateral flow assay test strip for detecting a target analyte in a test sample, the lateral flow assay test strip comprising: a sample receiving zone for receiving the test sample; a reaction zone overlapping with or downstream from the sample receiving zone, the reaction zone comprising: a conjugatively-labeled detector reagent that specifically binds to the target analyte, the conjugatively-labeled detector reagent having an analyte-specific label covalently-conjugated to an analyte binding reagent, and a labeled control reagent having a control-specific label, wherein the analyte-specific label is of a larger size or a greater signal sensitivity than the control-specific label, and the analyte-specific label has a different detectable characteristic feature than the control-specific label; a detection zone downstream of the sample receiving zone and the reaction zone, the detection zone comprising: a test line region having an immobilized detector capture reagent that specifically binds to the target analyte, and a control line region having an immobilized control capture reagent that specifically binds to the labeled control reagent, wherein the control line region is downstream of the test line region within the detection zone; and an absorbent zone downstream of the detection zone for capturing excess test sample.

In an embodiment, the lateral flow assay test strip comprises two or more of the conjugatively-labeled detector reagents and the analyte binding reagent of each of the conjugatively-labeled detector reagents specifically binds to a different target analyte.

In an embodiment, the detection zone comprises two or more of the test line regions and the immobilized detector capture reagent of each of the test line regions specifically binds to one of the different target analytes. In an embodiment, the detection zone comprises two or more of the test line regions and the immobilized detector capture reagent of each of the test line regions is the same. In an embodiment, the detection zone further comprises an autoantibody binding line having an immobilized anti-human-IgM antibody for binding of an autoantibody in the test sample.

In an embodiment, the analyte binding reagent is an antibody.

In an embodiment, the analyte-specific label is a gold nanoparticle or a gold nanoshell. In an embodiment, the gold nanoparticle or the gold nanoshell is between about 100 nm and about 200 nm in size. In an embodiment, the gold nanoparticle or the gold nanoshell is about 150 nm in size.

In an embodiment, the control-specific label comprises a gold particle. In an embodiment, the gold particle is comprised within a structure that is less than 50 nm in size. In an embodiment, the labeled control reagent is a gold-biotin complex.

In an embodiment, the conjugatively-labeled detector reagent specifically binds to a cardiac marker. In an embodiment, the cardiac marker is N-terminal (NT)-pro hormone BNP (NT-proBNP), cardiac troponin, creatine kinase (CK), CK-MB, myoglobin (Mb), lactate dehydrogenase (LDH), aspartate transaminase (AST), ischemia-modified albumin (IMA), pro-brain natriuretic protein, glycogen phosphorylase isoenzyme BB, or any combination thereof. In an embodiment, the cardiac marker is NT-proBNP.

In an embodiment, the different detectable characteristic feature between the analyte-specific label and the control-specific label is a different color signal. In an embodiment, the test line region is a blue or black color when the analyte-specific label localizes thereto and the control line region is a pink or red color when the control-specific label localizes thereto.

According another aspect of this disclosure, there is provided a lateral flow assay test device comprising one or more lateral flow assay test strips as described herein, and a housing.

In an embodiment, the housing comprises one or more quantification markers aligned beside the test line region, the control line region, or any combination thereof. In an embodiment, the one or more quantification markers are of a similar or identical color to the test line region when the analyte-specific label localizes thereto and the control line region when the control-specific label localizes thereto. In an embodiment, the housing comprises a QR code.

According to another aspect of this disclosure, there is provided a method for detecting a target analyte in a test sample, the method comprising: providing the lateral flow assay test strip as described herein or the lateral flow assay device as described herein; contacting a test sample with the sample receiving zone; allowing the test sample to migrate from the sample receiving zone to the absorbent zone; and determining or evaluating any detectable signal at the test line region and/or the control line region, to detect for the presence of the target analyte in the test sample, wherein the detectable signal is different at the test line region than at the control line region.

In an embodiment, the step of determining or evaluating the detectable signal at the test line region and/or the control line region comprises a comparison to one or more quantification markers aligned beside the test line region, the control line region, or any combination thereof. In an embodiment, the step of determining or evaluating the detectable signal at the test line region comprises assessing a test line signal intensity to determine a concentration of the target analyte in the test sample. In an embodiment, the step of determining or evaluating the detectable signal at the test line region further comprises assessing a control line signal intensity of the detectable signal at the control line.

In an embodiment, the step of assessing the test line signal intensity comprises a comparison between the test line signal intensity and the control line signal intensity, and optionally normalization of one or both to the one or more of the quantification markers.

In an embodiment, the step of determining or evaluating the detectable signal at the test line region and/or the control line region comprises measuring the detection zone or a portion thereof with a reader and providing a result using a data analyzer. In an embodiment, the reader is a computing device with a camera or other scanning capability. In an embodiment, the computing device is a portable device or a stationary device, each with a camera or other scanning capability. In an embodiment, the reader is a mobile phone with a camera. In an embodiment, the data analyzer is a software application or executable code stored on the computing device (e.g. mobile phone with a camera). In an embodiment, the data analyzer or the reader comprises an artificial intelligence (AI) engine having a trained AI model estimate a level of concentration of the target analyte in the test sample. In an embodiment, the data analyzer is configured for receiving and storing a patient's information. In an embodiment, the reader is configured for scanning a QR code to obtain information regarding the lateral flow test device or instruction regarding using the lateral flow test device.

In an embodiment, the AI engine is configured to differentiate a color intensity of the detectable signal at the test line region and the control line region and to provide one or more ranges of estimated numbers of the target analyte in the test sample to detect the accurate level of concentration of the target analyte in the test sample. In an embodiment, the AI model is trained using historical datasets to provide ranges of estimated numbers of the target analyte and to detect the level of concentration of the target analyte in the test sample.

In an embodiment, wherein the step of determining or evaluating the detectable signal at the test line region and/or the control line region further comprises establishing a standard range of numbers for the target analyte and classifying the level of concentration of the target analyte in the test sample. In an embodiment, wherein the standard range for the target analyte is established as low level, normal level, abnormal level, or high level and the level of concentration of the target analyte in the test sample is classified as low level, normal level, abnormal level, or high level.

In an embodiment, the method further comprises a step of communicating the result to a healthcare provider, an emergency assistance personnel to aid in care or to improve a pharmacogenomics analysis pool.

According to another aspect of this disclosure, there is provided a system for performing the method as described herein, the system comprising: the lateral flow assay test strip as described herein or the lateral flow assay device as described herein; a reader programmed to obtain one or more color intensity and/or light intensity measurements from one or more detectable signals; and a data analyzer comprising a software that identifies the color intensity and/or light intensity measurements obtained from the reader, and computes at least one parameter from the identified color intensity and/or light intensity measurements to determine the concentration of the target analyte in the test sample and provide a result.

In an embodiment, the data analyzer or the reader is machine learning trained or artificial intelligence trained to detect an accurate level of concentration of the target analyte in the test sample and provide a result. In an embodiment, the reader is a mobile phone with a camera.

In an embodiment, the reader is configured for scanning a QR code to obtain information regarding the lateral flow assay test strip or the lateral flow assay device, or to provide instruction regarding use of the lateral flow assay test strip or the lateral flow assay device.

In an embodiment, the data analyzer is configured for receiving and storing a patient's information. In an embodiment, the data analyzer is configured for providing evaluations using the patient's information stored and the result provided.

In an embodiment, the system further comprises an application-programming interface (API) configured for receiving the result from the data analyzer and sending the result to the software.

In an embodiment, the AI engine is configured to differentiate a color intensity of the detectable signal at the test line region and the control line region and to provide one or more ranges of estimated numbers of the target analyte in the test sample to detect the level of concentration of the target analyte in the test sample. In an embodiment, the AI model is trained using historical datasets to provide ranges of estimated numbers of the target analyte and to detect the level of concentration of the target analyte in the test sample.

In an embodiment, the system further comprises an established standard range for the target analyte and a classified level of concentration of the target analyte in the test sample. In an embodiment, the standard range for the target analyte is established as low level, normal level, abnormal level, or high level and the level of concentration of the target analyte in the test sample is classified as low level, normal level, abnormal level, or high level.

In an embodiment, the AI engine is configured to provide a color change grading, optimization of one or more lights of the camera, and optimization of one or more surrounding lights.

According to another aspect of this disclosure, there is provided a kit for detecting a target analyte in a test sample, the kit comprising: the lateral flow assay test strip as described herein or the lateral flow assay device as described herein; and instructions for using the test strips or the device for detecting the target analyte in the test sample.

In one embodiment, the kit further comprises a test sample extraction apparatus, an incubation buffer, a reaction mixture, a vial, or any combination thereof.

Other aspects and feature of the present disclosure will become apparent to those ordinarily skilled in the art upon review of the following description of specific embodiments.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 is a block diagram showing an exemplary lateral flow assay test strip according to one embodiment of this disclosure;

FIG. 2 is a block diagram showing an exemplary lateral flow assay test strip with multiple test line regions, according to some embodiments of this disclosure;

FIG. 3 is a block diagram showing an exemplary lateral flow assay test strip with an auto-antibody binding line, according to another embodiment of this disclosure;

FIG. 4 is a diagrammatic top view of an exemplary lateral flow device comprising a housing for containing a test strip, according to one embodiment of this disclosure;

FIG. 5 is a diagrammatic top view of another exemplary lateral flow device comprising a housing for containing multiple test strips, according to another embodiment of this disclosure;

FIG. 6 is a flowchart showing the steps of a process for detecting a target analyte in a test sample, according to one embodiment of this disclosure;

FIG. 7 is a schematic diagram showing the structure of a deep neural network (DNN) used in the process shown in FIG. 6.

FIG. 8 is a flowchart showing the steps of a process for detecting a target analyte in a test sample involving a reader and a data analyzer, according to one embodiment of this disclosure; and

FIG. 9 is a schematic diagram showing a lateral flow assay testing system, according to one embodiment of this disclosure.

### DETAILED DESCRIPTION

The present disclosure relates to lateral flow assay test strips, devices, systems, kits, and methods for detecting one or more target analytes in a test sample. The strips, devices, systems, and kits comprise improved design and features.

Advantageously, the lateral flow test strips disclosed herein comprise a conjugatively-labeled detector reagent and a labeled control reagent. The label of the labeled control reagent need not be covalently-conjugated, and in some embodiments it is not. Also advantageously, the conjugatively-labeled detector reagent and the labeled control reagent have different labels, wherein the analyte-specific label of the conjugatively-labeled detector reagent is of a larger size or a greater signal sensitivity than the control-specific label of the labeled control reagent, and the analyte-specific label has a different detectable characteristic feature than the control-specific label.

In some embodiments of the present disclosure, the different detectable characteristic feature between the analyte-specific label and the control-specific label is a different color signal. Advantageously, in these embodiments, the test line region and the control line region are different colors when the analyte-specific label and the control-specific label localize thereto. In an embodiment, the test line region is a blue or black color when the analyte-specific label localizes thereto and the control line region is a pink or red color when the control-specific label localizes thereto.

### Test Strips, Devices and Kits

In an embodiment, the present disclosure relates to a lateral flow assay test strip for detecting a target analyte in a test sample, the lateral flow assay test strip comprising: a sample receiving zone for receiving the test sample; a reaction zone overlapping with or downstream from the sample receiving zone, the reaction zone comprising: a conjugatively-labeled detector reagent that specifically binds to the target analyte, the conjugatively-labeled detector reagent having an analyte-specific label covalently-conjugated to an analyte binding reagent, and a labeled control reagent having a control-specific label, wherein the analyte-specific label is of a larger size or a greater signal sensitivity than the control-specific label, and the analyte-specific label has a different detectable characteristic feature than the control-specific label; a detection zone downstream of the sample receiving zone and the reaction zone, the detection zone comprising: a test line region having an immobilized detector capture reagent that specifically binds to the target analyte, and a control line region having an immobilized control capture reagent that specifically binds to the labeled control reagent, wherein the control line region is downstream of the test line region within the detection zone; and an absorbent zone downstream of the detection zone for capturing excess test sample.

As used herein, the term “target analyte” refers to any compound, molecule, or substance which is desired to be detected in a test sample. Examples of target analytes include, without limitation, an antigen, a protein, a peptide, a toxin, a drug, a metabolite, an antibody, a hormone, a vitamin, a cytokine, a microorganism (e.g. bacteria, virus, or protozoa), or the like. In an embodiment, the target analyte is a cardiac marker. Herein, “cardiac marker” refers to any compound, molecule, or substance that may be used to evaluate heart or cardiac function. Without limitation, the cardiac marker may be N-terminal pro B-type natriuretic peptide (NT-proBNP), cardiac troponin, D-Dimer, creatine kinase (CK), CK-MB, myoglobin (Mb), lactate dehydrogenase (LDH), aspartate transaminase (AST), ischemia-modified albumin (IMA), pro-brain natriuretic protein, glycogen phosphorylase isoenzyme BB, or any combination thereof. In an embodiment, the cardiac marker is NT-proBNP. The test sample may, for example, be any bodily fluid, such as blood, serum, plasma, or urine. In an embodiment, the target analyte includes hormones, cytokines and vitamins specifically relating to women health and wellness.

As used herein, by “sample receiving zone” it is meant the region of the lateral flow assay test strip where the test sample is deposited for fluid flow along the strip. The sample receiving zone may be of any size or shape. The sample receiving zone is located downstream in a fluid flow path from the detection zone and the absorbent zone. The sample receiving zone may overlap with or be located downstream of the reaction zone. In an embodiment, the sample receiving zone is located downstream of the reaction zone. By “downstream”, it is meant to refer to a location earlier in the fluid flow path than one or more other locations. The sample receiving zone may be made of any suitable material to receive the test sample and permit fluid flow along the test strip, e.g. by capillary action. In an embodiment, the sample receiving zone is comprised of a nitrocellulose membrane. Those skilled in the art will appreciate that various alternative materials are readily available.

The lateral flow test strips of the present disclosure comprise a reaction zone. As used herein, the “reaction zone” is intended to refer to a region of the test strip in which the test sample is able to come into contact with the conjugatively-labeled detector reagent. The reaction zone typically also includes the labeled control reagent for proper functioning of the control line by confirming fluid flow from the reaction zone to the region of the test strip where the control line is located. The reaction zone may be in the same region of the test strip as the sample receiving zone, or may partially overlap with the sample receiving zone, or may be entirely upstream along a fluid flow path from the sample receiving zone. In an embodiment, the conjugatively-labeled detector reagent and labeled control reagent are contained on the test strip as provided to an end-user. In other embodiments, the end-user may deposit the conjugatively-labeled detector reagent and labeled control reagent onto the test strip, for example shortly before a test sample is applied to the test strip. The sample receiving zone may be made of any suitable material suitable for receiving and/or maintaining in place the conjugatively-labeled detector reagent and labeled control reagent, and for permitting fluid flow of these reagents along the test strip, e.g. by capillary action. In an embodiment, the sample receiving zone is comprised of a nitrocellulose membrane. Those skilled in the art will appreciate that various alternative materials are readily available.

As used herein, the term “conjugatively-labeled detector reagent” refers to a reagent having a label covalently-conjugated thereto. In an embodiment, the reagent is any compound, molecule, or substance that is capable of specifically binding to a target analyte. In an embodiment and without limitation, the reagent is a protein, a peptide, an antibody, an aptamer, or an antibody mimetic. In a particular embodiment, the reagent is an antibody.

The label that is covalently-conjugated to the conjugatively-labeled detector reagent is referred to herein as the analyte-specific label. It may be any label suitable for use on a lateral flow assay test strip, so long as it is of a larger size or a greater signal sensitivity than the label used for the labeled control reagent. The label is a substance that has a detectable characteristic feature that may be identified and detected on the test strip, and distinguished from other elements or labels on the test strip. In some embodiments, the label has a distinctive optical property. In an embodiment, the analyte-specific label is a gold nanoparticle or a gold nanoshell. In an embodiment, the gold nanoparticle or the gold nanoshell is between about 100 nm and about 200 nm in size. In an embodiment, the gold nanoparticle or the gold nanoshell is about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in size. In an embodiment, the gold nanoparticle or the gold nanoshell is about 150 nm in size.

As used herein, the term “labeled control reagent” refers to a reagent having a label, in which the reagent does not bind to the target analyte (nor does the label). In an embodiment, the reagent is any compound, molecule, or substance that is not capable of binding to the target analyte. In an embodiment and without limitation, the reagent is a protein, a peptide, an enzyme, a vitamin, or an inert compound. In a particular embodiment, the reagent is biotin. In an embodiment, the label is not covalently-conjugated to the reagent for the labeled control reagent. In an embodiment, the label is attached or associated with the regent of the labeled control reagent by way of an ionic bond, hydrophobic interaction, hydrogen bond, Van der Waals forces, electrostatic interaction, or other suitable form of non-covalent bond or interaction.

The label of the labeled control reagent is referred to herein as the control-specific label. It may be any label suitable for use on a lateral flow assay test strip, so long as it is of a smaller size or a lesser signal sensitivity than the label used for the conjugatively-labeled detector reagent. In an embodiment, the label is a gold particle. In an embodiment, the gold particle is comprised within a structure that is less than 50 nm in size. In an embodiment, the gold particle is comprised within a structure that is about 50 nm, about 45 nm, about 40 nm, about 35 nm, about 30 nm, about 25 nm, or less in size. In an embodiment, the labeled control reagent is a gold-biotin complex.

An advantageous aspect of the lateral flow test strips of the present disclosure is that the analyte-specific label is of a larger size or a greater signal sensitivity than the control-specific label. In a particular embodiment, analyte-specific label is of a larger size than the control-specific label. This feature may be of a particular advantage for target analytes that are at a low abundance in the test sample. By having an analyte-specific label of a larger size or greater signal sensitivity than the control-specific label, it improves the ability to obverse a positive result at the test line without inferring background or control signal.

The lateral flow test strips of the present disclosure comprise a detection zone. As used herein, the “detection zone” is intended to refer to a region of the test strip in which, by fluid flow, the conjugatively-labeled detector reagent and labeled control reagent travel to and interact with the immobilized capture reagents on the test line region and the control line region. Within the detection zone, the control line region is located downstream of the test line region. As used herein, the term “immobilized capture reagent” refers to any compound, molecule, or substance that is immobilized onto the test line region (an immobilized detection capture reagent) or the control line region (an immobilized control capture reagent).

The immobilized detection capture reagent is capable of specifically binding to the target analyte in order to sandwich the target analyte between the conjugatively-labeled detector reagent and the immobilized detection capture reagent. Specific binding of the target analyte to the immobilized detection capture reagent localizes the conjugatively-labeled detector reagent at the test line region, thereby generating a positive signal regarding the presence of the target analyte. In an embodiment and without limitation, the immobilized detection capture reagent is a protein, a peptide, an antibody, an aptamer, or an antibody mimetic. In a particular, embodiment, the reagent is an antibody.

The immobilized control capture reagent is capable of specifically binding to the labelled control reagent. Specific binding of the labelled control reagent to the immobilized detection control reagent localizes the labelled control reagent at the control line region, thereby generating a positive control signal, indicating that the lateral flow assay test strip is functioning properly. In an embodiment, the immobilized control capture reagent is streptavidin.

In an embodiment of the lateral flow test strips of the present disclosure, the test strips comprise only a single test line region in the detection zone, with the control line region located downstream thereof. In an embodiment of such aspects having only a single test line region, the test line region comprises immobilized detection capture reagent for only a single target analyte. In other embodiments of such aspects having only a single test line region, the test line region comprises two or more different immobilized detection capture reagents, each immobilized detection capture reagent specific for a different target analyte. When the test strip comprises immobilized detection capture reagents specific for different target analytes, the test strips may also comprise two or more of the conjugatively-labeled detector reagents, wherein the analyte binding reagent of each of the conjugatively-labeled detector reagents specifically binds to a different target analyte.

In an embodiment of the lateral flow test strips of the present disclosure, the test strips comprise two or more test line regions in the detection zone, with the control line region located downstream of all of the test line regions. In an embodiment of such aspects having two or more test line regions, each test line region comprises an immobilized detection capture reagent that specifically binds a different single target analyte. Thus, in such embodiments, the test strips may be used to detect for the presence of two or more different target analytes. When the test strip comprises immobilized detection capture reagents specific for different target analytes, the test strips may also comprise two or more of the conjugatively-labeled detector reagents, wherein the analyte binding reagent of each of the conjugatively-labeled detector reagents specifically binds to a different target analyte. The different conjugatively-labeled detector reagents may also have different labels so as to generate a different detectable characteristic (e.g. color) at each test line region. Alternatively, the different conjugatively-labeled detector reagents may each have the same label so as to generate the same detectable characteristic (e.g. color) at each test line region.

In other embodiments of aspects herein having two or more test line regions, each test line region comprises immobilized detection capture reagent for the same target analyte. Thus, in such embodiments, the test strips may be used to detect for the presence of a single target analyte, and the signal intensities at each test line region may be used for quantification purposes.

In an embodiment, the detection zone further comprises an autoantibody binding line. The autoantibody binding line may be used, for example, as an internal control for quantification purposes or as another positive control indicator of a properly functioning test strip or a properly loaded test sample. In an embodiment, the autoantibody binding line has an immobilized anti-human-IgM antibody.

The lateral flow test strips of the present disclosure comprise an absorbent zone. As used herein, the “absorbent zone” is intended to refer to a region of the test strip for capturing excess test sample. The absorbent zone is located downstream of the detection zone. The absorbent zone may be comprised of the same material as the other zones as described herein, or may be comprised of a material having increased absorbency. Those skilled in the art will appreciate that various alternative materials are readily available.

In another aspect, the present disclosure relates to a lateral flow assay test device comprising one or more lateral flow assay test strips as described herein and a housing.

In an embodiment, the lateral flow assay test device comprises a single lateral flow assay test strip. In an embodiment, the lateral flow assay test device comprises two or more lateral flow assay test strips. In an embodiment, when the lateral flow assay test device has two or more lateral flow assay test strips, each is for detecting a different target analyte. In this configuration, a single lateral flow assay test device may be used to determine the presence of two or more different target analytes in a test sample. In an embodiment, when the lateral flow assay test device has two or more lateral flow assay test strips, each is for detecting the same target analyte. In this configuration, each test strip in a single lateral flow assay test device may be identical and used to confirm the results of the other, or each test strip may be different and may, for example, be used for comparison for better quantification of the target analyte in the test sample.

The lateral flow assay test device comprises a housing. The housing may be any structure to hold the test strips. In an embodiment, the housing is a plastic casing.

The housing may comprise one or more quantification markers aligned beside the test line region, the control line region, or any combination thereof. The quantification markers may be used as a standard to which the intensity of the signals generate at the test line region and the control line region are compared.

The housing may comprise one or more QR codes. The QR codes may be used for any suitable purpose, such as for providing instructions on use, for providing information on interpreting results, for storing information regarding the test strip (e.g. batch number, target analyte specificity, and/or the like).

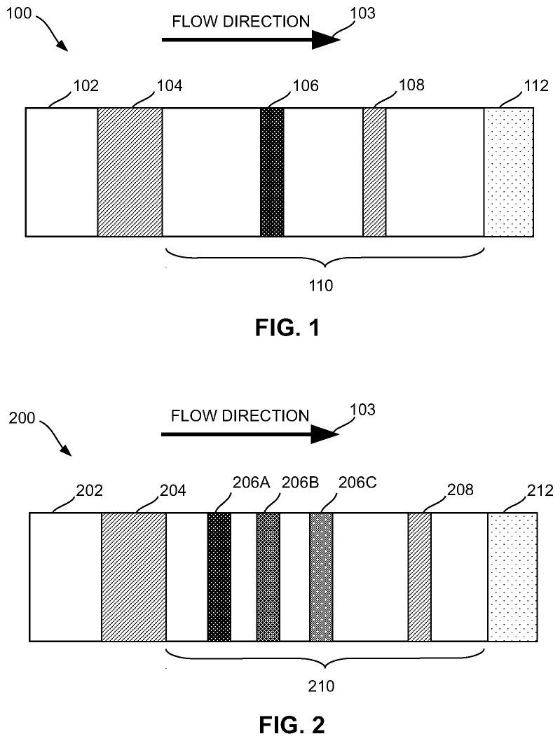

Turning now to FIG. 1, a lateral flow assay test strip is generally identified using reference numeral 100. The test strip 100 in this embodiment, from upstream to downstream according to the fluid flow direction (indicated by the arrow 103), comprises a sample receiving zone 102, a reaction zone 104, a detection zone 110, and an absorbent zone 112. These zones are as described herein and typically made of materials that allow fluid to flow, by capillary action, from the sample receiving zone 102 to the absorbent zone 112 (e.g. nitrocellulose membrane). Those skilled in the art will appreciate that various alternative materials are readily available.

The sample receiving zone 102 is located at the upstream end of the test strip for receiving a test sample. The reaction zone 104, located downstream of the sample receiving zone 102 or overlapping with the sample receiving zone 102, is made of the same material or different material from the sample receiving zone 102. The reaction zone 104 comprises a conjugatively-labeled detector reagent (not shown) as described herein that will specifically bind to a target analyte of interest that may or may not be present in the test sample. The conjugatively-labeled detector reagent has an analyte-specific label covalently-conjugated to an analyte binding reagent (not shown) as described herein.

There are generally two methods of preparing a labeled detector reagent, namely non-covalent and covalent conjugation. Non-covalent interaction does not provide a strong bond and is substantially less stable than covalent conjugation or coupling. A covalent conjugation or coupling permanently immobilizes the molecules of interest to label particles such as gold nanoparticles, with a linker such as N-hydroxysuccinimide (NHS), carboxyl groups, or amine groups. This type of interaction is stronger and provides improved stability compared to non-covalent interactions.

Using a covalently-labeled detector reagent, where an analyte-specific label (e.g. gold nanoshells) is covalently-conjugated to an analyte binding reagent (e.g. an antibody), provides the ability to control the concentration. This provides a controlled usage of antibodies and provides a high degree of sensitivity to the assays, and avoids the need for secondary reagents for analyte detection.

The reaction zone 104 further comprises a labelled control reagent having a control-specific label (not shown) as described herein. Unlike the covalently-labeled detector reagent, the control-specific label may be non-covalently coupled to the control reagent and does not need to be a covalent conjugation.

The analyte-specific label is of a larger size or a greater signal sensitivity than the control-specific label, and the analyte-specific label has a different detectable characteristic feature (e.g. color) than the control-specific label. For example, the analyte-specific label may be a gold nanoparticle or a gold nanoshell having a size between about 100 nm and about 200 nm, and the control-specific label may be a gold particle, such as a gold-biotin complex, that is comprised in a structure less than 50 nm in size. Further, the detectable characteristic feature (e.g. color) of a gold nanoshell label for the analyte-specific label may be one color (e.g. a blue or black color) and is different from the detectable characteristic feature of the gold-biotin (e.g. a pink or red color). This will result in different color signals at the test line region(s) 106 than at the control line region 108 on the test strip. In this manner, it is advantageous for ease of visual distinction, reduction in background, and quantification.

The detection zone 110, downstream of the sample receiving zone 102, comprises a test line region 106 and a control line region 108 downstream of the test line region 106. The test line region 106 contains immobilized detector capture reagents (not shown) as described herein that specifically bind to the target analyte. If the target analyte is present in the test sample, the conjugatively-labeled detector reagent will bind with the target analyte, travel through the detection zone 110, and be sandwiched by the immobilized detector capture reagents at the test line region 106, thereby displaying a test line signal indicating the presence of the target analyte in the test sample.

The control line region 108 contains immobilized control capture reagents (not shown) as described herein that specifically bind to the labeled control reagent. When the labeled control reagents travel through the detection zone 110, they will be immobilized and stop at the control line region 108, thereby displaying a control line signal indicating that the test strip is functioning properly. The control line signal has a different detectable characteristic feature than the test line signal.

The absorbent zone 112 is at the downstream end of the test strip and will capture any excess fluid flow.

Turning now to FIG. 2, a lateral flow assay test strip is generally identified using reference numeral 200. The test strip 200 in this embodiment, is generally similar to the test strip 100, and comprises a sample receiving zone 202, a reaction zone 204, a detection zone 210 with multiple test line regions, and an absorbent zone 112.

In an embodiment, the reaction zone 204 comprises multiple conjugatively-labeled detector reagents capable of binding to different target analytes suspected to be present in a test sample. The detection zone 210 contains multiple corresponding test line regions 206A to 206C containing immobilized detector capture reagents that specifically bind to each one of the different target analytes. Therefore, in this embodiment, the appearance or absence of multiple test line signals at test line regions 206A to 206C indicates the presence or absence of each one of the different target analytes.

Furthermore, the multiple different covalently-labeled detector reagents may have the same label (e.g. gold nanoshells) resulting in multiple test line signals with the same detectable characteristic feature (e.g. color), or may have different labels with different detectable characteristic features. Those skilled in the art will appreciate that various alternative embodiments and labels are readily available for those detector reagents.

In another embodiment, the reaction zone 204 comprises one conjugatively-labeled detector reagent capable of binding to a specific target analyte that may be present in a test sample. The detection zone 210 contains multiple test line regions 206A to 206C containing immobilized detector capture reagents that specifically bind to the specific target analyte. Therefore, in this embodiment, if the specific target analyte is present, multiple test line signals at test line regions 206A to 206C will appear in the detection zone 210 but with different signal intensities. For example, in an embodiment, test line region 206A will be able to capture the highest concentration of conjugatively-labeled detector reagent target analyte complex and will show the strongest signal and detectable characteristic feature (i.e. brightest or darkest color) compared to the test line signals at 206B and 206C. This embodiment allows the test strip to provide both qualitative and quantitative results of the presence and concentration of the target analyte in a test sample. Furthermore, the appearance or absence of downstream test line signals, i.e. 206 B and/or 206C, will further quantitatively determine the concentration of the target analyte and will provide a more accurate determination compared to the embodiment with only one test line region.

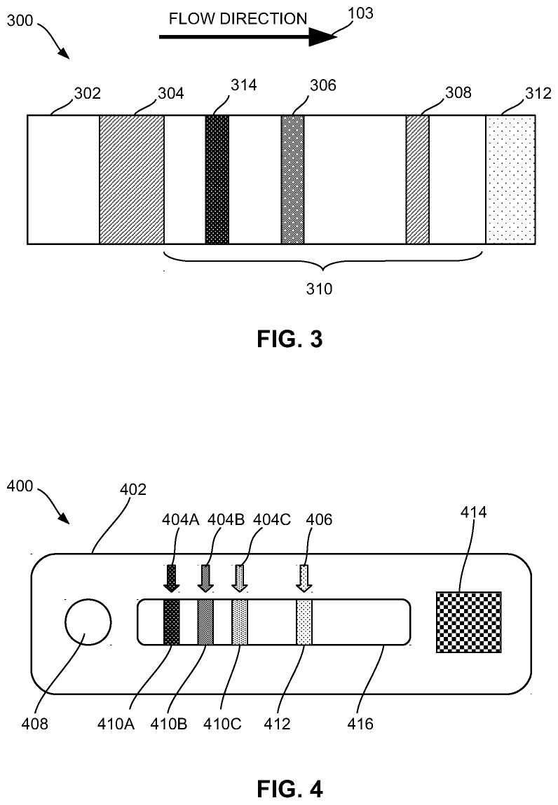

Turning now to FIG. 3, a lateral flow assay test strip is generally identified using reference numeral 300. The test strip 300 in this embodiment is generally similar to the test strip 100, and comprises a sample receiving zone 302, a reaction zone 304, a detection zone 310, and an absorbent zone 312. Test strip 300 further comprises an autoantibody binding line 314 in the detection zone 310, in addition to the test line region 306 and the control line region 308. The autoantibody binding line 304 has immobilized anti-human-IgM antibody for binding to autoantibodies in the test sample.

Turning now to FIG. 4, a lateral flow assay test device is generally identified using reference numeral 400. The test device 400 comprising a housing 402, a test strip that is generally similar to the test strips 100, 200 and 300, an opening 408 exposing the sample receiving zone of the test strip, another opening or window 416 for exposing or showing the detection zone of the test strip for visual inspection of the signals, markers 404A-404C on the housing at respective locations for approximately indicating where each of the test lines will appear, marker 406 on the housing at a location for approximately indicating where the control line will appear, and a QR code 414 on the housing.

The housing 402 is used to receive the test strip and secure it in place, and may be made of any suitable materials (e.g. plastic). The opening, window, or hole 408 on the housing allows a fluid test sample to pass therethrough and directly contact the sample receiving zone of the test strip.

The opening or window 416 may be either an opening or window cut on the housing or made of clear transparent materials (e.g. glass or plastic) that allows visual inspection of the reaction zone of the test strip. In this embodiment, markers 404A-404C and 406 (e.g. colored arrows) are placed on the surface of the housing 402 to further aid visual inspection and quantification. The markers 404A-404C and 406 are placed at the approximate locations where the test line signals 410A-410C and the control line signal 412 are expected to appear. The markers may have various shapes (e.g. arrows or lines) and colors (e.g. colors corresponding to the expected color of the corresponding signals).

In some other embodiments, the markers are quantification markers (e.g. with fixed or set standard color intensity). The quantification markers may be used to compare with the test line and/or control line signals to determine the intensity of the signals. For example, a marker on the housing may be used as a defined standard color intensity. The intensity of the marker's color may be compared with the actual signal appearing on the test strip to determine if the concentration is more than or less than the defined standard.

The QR code 414 on the housing 402 may be scanned by a reader such as a mobile phone to access information relating to the test device and guidelines for using the test device. Furthermore, if the test strip is for a specific analyte (i.e. NT-proBNP or other cardiac marker) or relates to diagnosis of a specific disease (e.g. heart failure or COVID-19), the QR code also directs to helpful information relating to those, including laboratory contacts, physician contacts, or links to helpful websites.

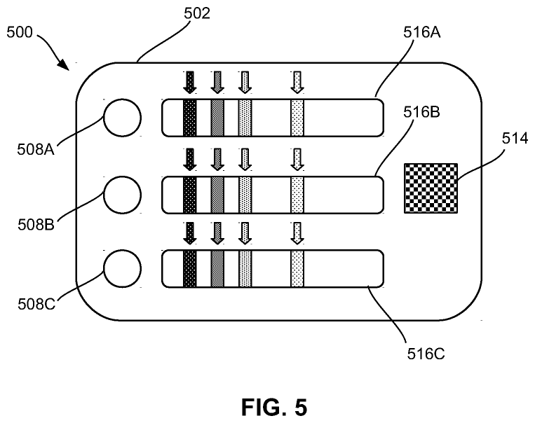

Turning now to FIG. 5, a lateral flow assay test device is generally identified using reference numeral 500. The test device 500 is generally similar to test device 400, but contains multiple test strips in one housing 502. The test device 500 contains multiple openings 508A-508C for receiving samples and multiple openings 516A-516C for visual inspection of the detection zones of the test strips and allows multiple tests to be completed using one device.

In one embodiment, each one of the multiple test strips may be used for the same target analyte or same set of target analytes (e.g. cardiac markers). In another embodiment, each of the multiple test strips may be used for different target analytes or different sets of target analytes, that together, may aid diagnosis of a certain disease or certain diseases. Those skilled in the art will appreciate that various alternative embodiments and combinations are readily available for those arrangements.

In another embodiment, the present disclosure relates to a kit for detecting a target analyte in a test sample, wherein the kit comprises the lateral flow assay test strip as described herein or the lateral flow assay device as described herein, and instructions for using the test device for detecting the target analyte in the test sample.

In an embodiment, the kit may further comprise a test sample extraction apparatus, an incubation buffer, a reaction mixture, a vial, or any combination thereof.

### Systems and Methods

In an embodiment, the present disclosure relates to a method for detecting a target analyte in a test sample, the method comprising: (a) providing the lateral flow assay test strip as described herein or the lateral flow assay device as described herein; (b) contacting a test sample with the sample receiving zone; (c) allowing the test sample to migrate from the sample receiving zone to the absorbent zone; and (d) determining or evaluating any detectable signal at the test line region and/or the control line region, to detect for the presence of the target analyte in the test sample, wherein the detectable signal is different at the test line region than at the control line region.

In an embodiment, the present disclosure relates to a system for performing the method as described herein, the system comprising: the lateral flow assay test strip as described herein or the lateral flow assay device as described herein; a reader programmed to obtain one or more color intensity and/or light intensity measurements from one or more detectable signals; and a data analyzer comprising computer-executable code or instructions for identifying the color intensity and/or light intensity measurements obtained from the reader and computing at least one parameter from the identified color intensity and/or light intensity measurements to determine the concentration of the target analyte in the test sample and provide a result.

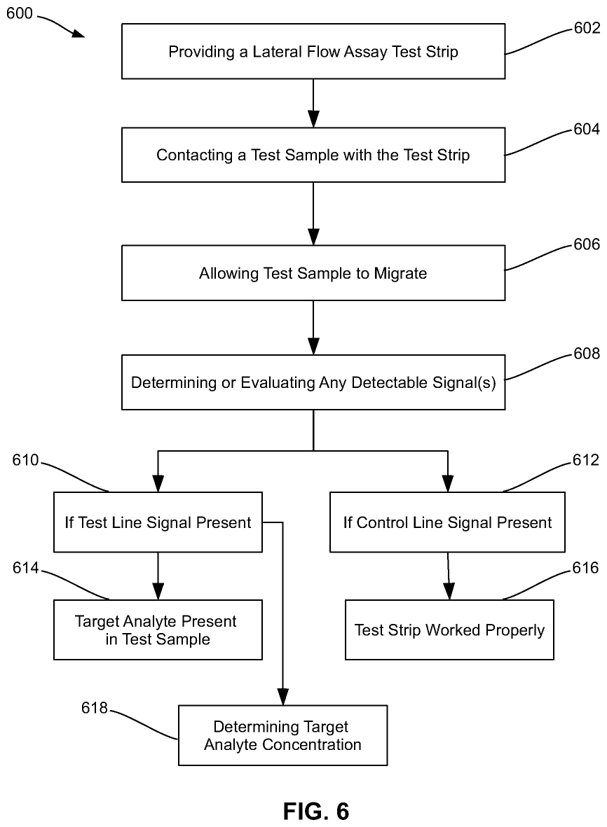

FIG. 6 is a flowchart showing the steps of a method 600 for detecting a target analyte in a test sample, according to one embodiment of this disclosure. The method 600 begins when a lateral flow assay test strip is provided (step 602). At step 604, a test sample is contacted with the test strip. For example, dropping one or more drops of the fluid test sample onto the sample receiving zone 102 of the test strip 100 or to the opening 408 of the test device 400 (if the test strip 100 has been inserted into the test device 400).

At step 604, the test sample is allowed to migrate (e.g. by capillary movement across the nitrocellulose membrane) through the test strip from the sample receiving zone 102 towards the absorbent zone 112. During step 604, if the target analyte is present in the test sample, the conjugatively-labeled detector reagent, with an analyte-specific label, will specifically bind to the target analyte and will be immobilized and captured at the test line region 106 by the immobilized detector capture reagent binding to the target analyte. The labeled control reagent, with a control-specific label, will stop and be immobilized at the control line region 108 by the immobilized control capture reagent that binds to the labeled control reagent.

At step 608, the test line region(s) and the control line region are visually inspected by human eye or detected and measured by a reader, to determine or evaluate if there is any detectable test line signal(s) or control line signal.

At steps 610 and 612, the presence of any test line signal and any control line signal is determined. This can be done by visual inspection by human eyes or by a reader (e.g. any computing device with a camera). For example, visual inspection and comparison of any test line and/or control signal(s) appearing within the detection zone of the test strip with corresponding markers on the housing 402 of the device 400 can be conducted. The markers will indicate the approximate location(s) of where the signals will appear for their corresponding signals.

In other embodiments, for improved accuracy and sensitivity (e.g. when signals are difficult to be caught by human eyes), a reader may be used in combination with a data analyzer, such as a software application or executable code stored on a computing device with a camera or other scanning capability, to measure and detect any signals and to evaluate and determine the presence of a test line signal and/or a control line signal.

If there is a control line signal present, then this indicates the test strip worked properly (step 616). If there is a test line signal present, then this indicates that the target analyte is present in the test sample (step 614).

At step 618, if quantitative analysis is desired, the target analyte concentration is determined by assessing any test line signal intensity. Again, this can be done by a visual inspection by human eyes or by a reader. The markers on the housing 402 of the device 400 may be quantification markers (e.g. with fixed or defined standard color intensity). A comparison may be conducted to determine whether the signal(s) has a stronger or weaker intensity compared to its corresponding quantification marker(s), thus allowing determination of the concentration of the target analyte. For improved accuracy and sensitivity, a reader may be used in combination of a data analyzer to detect, measure, and evaluate the signals to determine their intensity and thus the concentration of the target analyte.

Furthermore, an additional optional step (not shown) of comparing the test line signal intensity and the control line signal intensity for normalization of the signals to the quantification markers can also be conducted prior to step 618.

In some embodiments, the data analyzer or the reader may use an artificial intelligence (AI) engine such as a machine learning engine to more accurately detect the level of concentration of the target analyte based on the test line signal intensity.

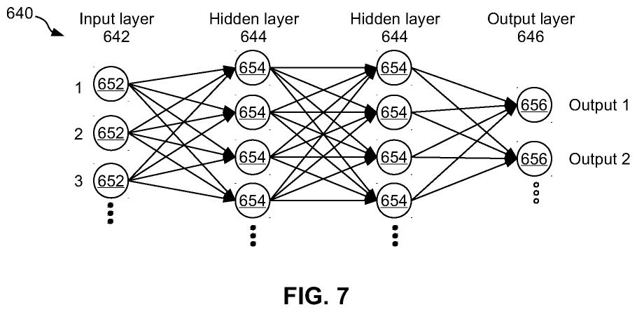

In these embodiments, the AI engine comprises an AI model such as a deep neural network (DNN). FIG. 7 is a schematic diagram of a DNN 640. As shown, the DNN 640 comprises an input layer 642 having one or more input nodes 652, one or more hidden layers 644 each having a plurality of intermediate nodes 654, and an output layer 646 having one or more output nodes 656. The input nodes 652 are configured for receiving the image of the window 416 and the markers 404 of the test device 400. Each intermediate node 654 of the first hidden layer combines the outputs of the nodes in its previous layer (being the nodes 652 of the input layer 642 or the nodes 654 of a previous hidden layer 644) for obtaining or extracting a set of features from its previous layer, and each output node 656 combines the intermediate nodes of the last hidden layer for generating estimation outputs such as ranges of estimated numbers of the target analyte in the test sample and the estimated level of concentration of the target analyte.

For example, the first hidden layer may obtain edges of graphic objects in the image received by the input layer 642, the second hidden layer may obtain graphic objects of markers 404, test line signals 410, and control line signals 412 from the edges output from the first hidden layer, the third hidden layer may obtain the color intensities of the markers 404, the test line signals 410, and the control line signals 412, and the forth hidden layer may obtain the ranges of estimated numbers of the target analyte in the test sample and the estimated level of concentration of the target analyte for the output layer 646 to output to the user.

The DNN 640 may be trained by using a plurality of historical data such as historical test line signal intensities, corresponding quantification marker intensities, and corresponding estimation outputs. The historical test line signal intensities and corresponding quantification marker intensities are input to the input nodes 652 of the DNN 640 to generate training estimation outputs which are then compared with the historical estimation outputs. The DNN 640 is then optimized based on the comparison between the training estimation outputs and the historical estimation outputs for minimizing the difference therebetween. After training, the DNN 640 is then used by the data analyzer or reader for estimating the level of concentration of the target analyte based on the test line signal intensity.

In an embodiment, the estimated level of concentration of the target analyte may be classified as a low, normal, abnormal, or high level to further aid the diagnosis of any disease.

In an embodiment, the DNN 640 also comprises necessary hidden layers 644 trained for adapting to distortions and variations in the input images such as various color change gradings, camera lighting conditions, ambient light conditions for improving the accuracy of the estimated output.

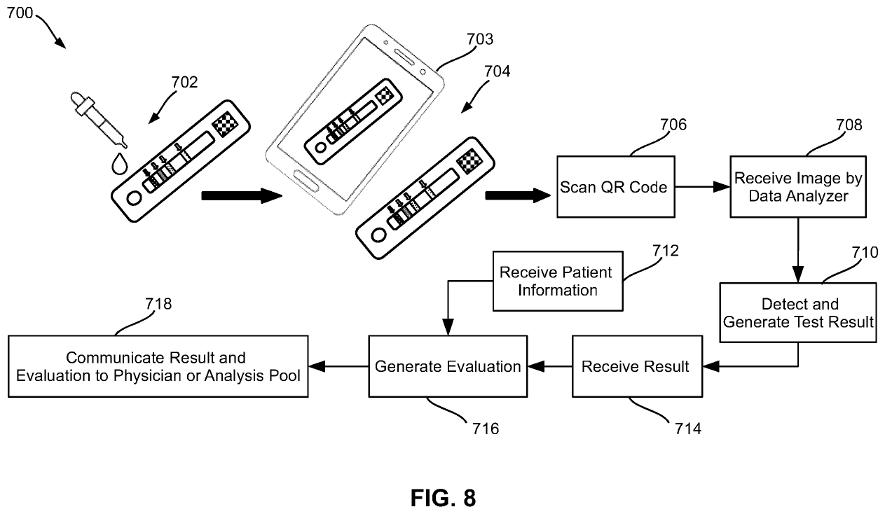

FIG. 8 is another flowchart showing the steps of a method 700 for detecting a target analyte in a test sample and then generating a result and an evaluation, according to one embodiment of this disclosure. Compared to FIG. 6, FIG. 8 focuses more on showing the steps of obtaining a result, generating an evaluation based on the result, and then communicating such with a physician for diagnosis purposes.

Similar to the method 600, method 700 also begins by contacting a test sample with the test device's sample receiving zone (step 702).

At step 704, a reader 703 (e.g. a mobile phone or a computer with a camera) is used to capture and read any signals from the test device (e.g. in the form of a picture for labels with visually detectable characteristic features).

At step 706, which may be performed before step 702, or before, after, or at the same time as step 704, the reader is used to scan a QR code (e.g. QR code 414 of the test device 400). This allows the user to access information and guidelines about using the test device and any further information relating to the specific target analyte or a specific disease.

The reader 703 may also be combined with a data analyzer, such as a software application stored in the non-transitory storage media (such as a memory) of a smartphone or computer and executed by a processor thereof to access, receive, and store the information provided by the QR code.

At step 708, the image taken by the reader 703 is received and stored by a data analyzer.

At step 710, the signals are detected and measured by the data analyzer and a test result is generated (e.g. the presence of a signal and the intensity of a signal). As described above, the reader 703 and/or the data analyzer may use an AI engine to more accurately detect the signals and their intensity. This feature is beneficial in particular situations when the analyte (i.e. cardiac markers) is very difficult to detect and requires a greater sensitivity.

At step 714, the result is received and stored by the data analyzer. An application-programming interface (API) configured for receiving such result may also be used to receive and store such result and then sending the result to a software application to generate an evaluation.

At step 712, before an evaluation is generated at step 716, patient information is inputted, received and stored by the software application. This will provide further useful information in addition to test results.

At step 716, the software application will gather all the information and results it received and generate an evaluation based on such (e.g. a preliminary diagnosis result indicating whether the patient has a heart failure or not).

At step 718, such evaluation will be communicated and sent to a healthcare provider (e.g. a physician) or an emergency assistance personnel to aid in care or to improve a pharmacogenomics analysis pool.

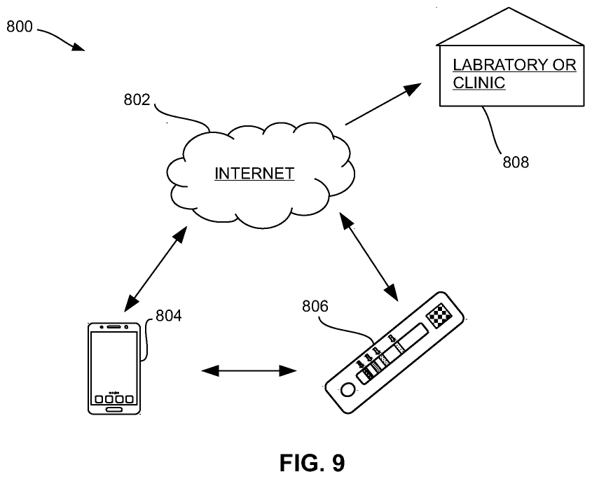

Turning now to FIG. 9, a lateral flow assay testing system is shown and is generally identified using reference numeral 800.

The system 800 comprises a lateral flow assay testing device 806 and a reader and data analyzer combination 804 (e.g. a smartphone with a software application) that comprises a wired or wireless networking module (not shown) to connect to a network 802 such as the Internet to receive relevant information such as the patient's information, and/or to send relevant information such as the testing result or evaluation to a physician or a person at a laboratory of a clinic 808.

When a testing result is obtained and an evaluation generated by the reader and data analyzer combination 804, this information may be immediately communicated to a physician or a clinic 808.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A lateral flow assay test strip for detecting a target analyte in a test sample, the lateral flow assay test strip comprising:
a sample receiving zone for receiving the test sample;
a reaction zone overlapping with or downstream from the sample receiving zone, the reaction zone comprising:
a conjugatively-labeled detector reagent that specifically binds to the target analyte, the conjugatively-labeled detector reagent having an analyte-specific label covalently-conjugated to an analyte binding reagent, and
a labeled control reagent having a control-specific label,
wherein the analyte-specific label is of a larger size or a greater signal sensitivity than the control-specific label, and the analyte-specific label has a different detectable characteristic feature than the control-specific label;
a detection zone downstream of the sample receiving zone and the reaction zone, the detection zone comprising:
a test line region having an immobilized detector capture reagent that specifically binds to the target analyte, and
a control line region having an immobilized control capture reagent that specifically binds to the labeled control reagent,
wherein the control line region is downstream of the test line region within the detection zone; and
an absorbent zone downstream of the detection zone for capturing excess test sample.

2. The lateral flow assay test strip of claim 1, which comprises two or more of the conjugatively-labeled detector reagents, wherein the analyte binding reagent of each of the conjugatively-labeled detector reagents specifically binds to a different target analyte.

3. The lateral flow assay test strip of claim 2, wherein the detection zone comprises two or more of the test line regions, and wherein the immobilized detector capture reagent of each of the test line regions specifically binds to one of the different target analytes.

4. The lateral flow assay test strip of claim 1, wherein the detection zone comprises two or more of the test line regions, and wherein the immobilized detector capture reagent of each of the test line regions is the same.

5. The lateral flow assay test strip of claim 1, wherein the detection zone further comprises an autoantibody binding line, the autoantibody binding line having an immobilized anti-human-IgM antibody for binding of an autoantibody in the test sample.

6. The lateral flow assay test strip of claim 1, wherein the analyte binding reagent is an antibody.

7. The lateral flow assay test strip of claim 1, wherein the analyte-specific label is a gold nanoparticle or a gold nanoshell.

8. The lateral flow assay test strip of claim 7, wherein the gold nanoparticle or the gold nanoshell is between about 100 nm and about 200 nm in size.

9. The lateral flow assay test strip of claim 7, wherein the gold nanoparticle or the gold nanoshell is about 150 nm in size.

10. The lateral flow assay test strip of claim 1, wherein the control-specific label comprises a gold particle.

11. The lateral flow assay test strip of claim 9, wherein the gold particle is comprised within a structure that is less than 50 nm in size.

12. The lateral flow assay test strip of claim 10, wherein the labeled control reagent is a gold-biotin complex.

13. The lateral flow assay test strip of claim 1, further comprising a housing for containing a test strip, wherein the shell comprises one or more markers beside the test line region and control line region.

14. The lateral flow assay test strip of claim 1, wherein the conjugatively-labeled detector reagent specifically binds to a cardiac marker.

15. The lateral flow assay test strip of claim 13, wherein the cardiac marker is NT-proBNP.

16. The lateral flow assay test strip of claim 1, wherein the different detectable characteristic feature between the analyte-specific label and the control-specific label is a different color signal.

17. The lateral flow assay test strip of claim 15, wherein the test line region is a blue or black color when the analyte-specific label localizes thereto and the control line region is a pink or red color when the control-specific label localizes thereto.

18. A lateral flow assay test device comprising one or more lateral flow assay test strips of claim 1 and a housing.

19. The lateral flow assay test device of claim 15, wherein the housing comprises one or more quantification markers aligned beside the test line region, the control line region, or any combination thereof.

20. The lateral flow assay test device of claim 18, wherein the housing comprises a QR code.

21.-49. (canceled)

50. A kit for detecting a target analyte in a test sample, comprising:
the lateral flow assay test strip of claim 1 or the lateral flow assay device of claim 18; and
instructions for using the lateral flow assay test strip or the lateral flow assay device for detecting the target analyte in the test sample.
